(19) United States
(12) Patent Application Publication
(10) Pub. No.: US 2012/0197112 A1
(43) Pub. Date: Aug. 2, 2012

(54) SPATIALLY-LOCALIZED OPTICAL COHERENCE TOMOGRAPHY IMAGING

(75) Inventor: Roger J. McNichols, Pearland, TX (US)

(73) Assignee: BIOTEX, INC., Houston, TX (US)

(21) Appl. No.: 13/361,946

(22) Filed: Jan. 30, 2012

Related U.S. Application Data
(60) Provisional application No. 61/437,675, filed on Jan. 30, 2011.

(51) Int. Cl. A61B 6/00

(52) U.S. Cl. 600/425

(57) ABSTRACT

This invention generally relates to devices and methods for optical coherence tomography (OCT), and also to devices and methods for spatially-localized OCT imaging. In one aspect, a spatially-localized OCT imaging system may include an OCT probe, a spatially-resolvable positioning device, a tracking system, and a data acquisition system. Some or all of the components may also be linked and/or otherwise combined.

### CROSS-REFERENCE TO RELATED APPLICATIONS

This application claims the benefit of U.S. provisional patent application Ser. No. 61/437,675, filed Jan. 30, 2011, entitled “SPATIALLY-LOCALIZED OPTICAL COHERENCE TOMOGRAPHY IMAGING”, the entire contents of which are hereby incorporated by reference.

### FIELD OF THE INVENTION

This invention relates to devices and methods for optical coherence tomography (OCT), and particularly to devices and methods for spatially-localized OCT imaging.

### BACKGROUND OF THE INVENTION

Optical Coherence Tomography (OCT) continues to be an emergent imaging technology occupying a unique place in the resolution-vs-depth-of-penetration continuum. Though it is widely used in ophthalmology, it has yet to find routine use in other fields. While imaging modalities such as ultrasound, MRI, and radiography can provide images of an entire anatomical field of interest, OCT can typically inform upon only a small region of interest. Thus, interpretation of OCT is typically dependent upon a registration to other imaging modalities or anatomic landmarks which can be difficult to obtain and has made clinical adoption in some settings difficult or impractical.

### SUMMARY OF THE INVENTION

This invention generally relates to devices and methods for optical coherence tomography (OCT), and also to devices and methods for spatially-localized OCT imaging. In one aspect, a spatially-localized OCT imaging system may include an OCT probe, a spatially-resolvable positioning device, a tracking system, and a data acquisition system. Some or all of the components may also be linked and/or otherwise combined. In general, any appropriate OCT probe may be utilized and may be selected, modified and/or otherwise tailored for a particular application(s). In some embodiments, other forms of imaging or sensing modalities may also be used instead of or in combination with OCT and may include, for example, fluorescence or spectroscopy. In some embodiments, a spatially-resolvable positioning device may include, for example, a magnetic field positioning probe. In an exemplary embodiment, a spatially-resolvable positioning device may be attached, integrated, and/or otherwise in a known orientation to an OCT probe such that tracking of the spatially-resolvable positioning device may be utilized to track the position of the OCT probe. The OCT probe may thus acquire data of a target, such as, for example, a region of tissue, and a data acquisition system may receive and/or store the data. The data may then be or simultaneously be synchronized to the tracked position of the OCT probe such that the data may be spatially-resolved. This may be desirable as the data acquired by an OCT probe may generally or in some cases be of a relatively small region of the target and/or difficult to resolve spatially in relation to the target as a whole.

In one exemplary aspect, the spatially-resolved data may be utilized to generate a three-dimensional (3D) dataset. This may be desirable as, for example, OCT probes typically gather two-dimensional (2D) data and it may be useful or desirable to resolve such data in 3D. In one exemplary embodiment, the tracking system and data acquisition system may collect and/or synchronize positioning data and OCT-gathered data temporally such that the data may be correlated to generate a spatially-resolved dataset, such as 3D dataset. The 3D dataset may further be utilized, for example, to generate a 3D graphical representation of the scanned target.

In another exemplary aspect, the tracking system may be utilized to guide acquisition of data from a 2D OCT probe to generate a 3D dataset. In one embodiment, the tracking system may, for example, indicate the region of a target for which data has been acquired. For example, the tracking system may indicate to a user what volume of space of the target for which data has been acquired which may enable the user to “fill in” the space during data acquisition to help ensure that data has been acquired for a desired volume.

The present invention together with the above and other aspects and advantages may best be understood from the following detailed description of the embodiments of the invention and as illustrated in the drawings.

### BRIEF DESCRIPTION OF THE FIGURES

FIG. 1 illustrates a spatially-resolved OCT system in an embodiment of the present invention;

FIG. 2 illustrates an OCT probe with a spatially-resolvable probe in another embodiment of the present invention; and

FIGS. 3-11 show examples of embodiments of the present invention.

### DETAILED DESCRIPTION OF THE INVENTION

The detailed description set forth below is intended as a description of the presently exemplified system, devices and methods provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified systems, methods, devices and materials are now described.

This invention generally relates to devices and methods for optical coherence tomography (OCT), and also to devices and methods for spatially-localized OCT imaging. In one aspect, a spatially-localized OCT imaging system may include an OCT probe, a spatially-resolvable positioning device, a tracking system, and a data acquisition system. Some or all of the components may also be linked and/or otherwise combined. In general, any appropriate OCT probe may be utilized and may be selected, modified and/or otherwise tailored for a particular application(s). In some embodiments, other forms of imaging or sensing modalities may also be used instead of or in combination with OCT and may include, for example, fluorescence or spectroscopy. The OCT probe may further be a 2D or a 3D OCT probe.

In some embodiments, a spatially-resolvable positioning device and/or tracking system may include, for example, a magnetic field positioning probe and system. For example, a six degree-of-freedom active positioning system, such as TrackSTAR DC magnetic field six degree-of-freedom active positioning system (trackSTAR, Ascension Technology, Burlington, Vt.) may be utilized. In an exemplary embodiment, a spatially-resolvable positioning device may be attached, integrated and/or otherwise in a known orientation to an OCT probe such that tracking of the spatially-resolvable positioning device may be utilized to track the position of the OCT probe. The OCT probe may thus acquire data of a target, such as, for example, a region of tissue, and a data acquisition system may receive and/or store the data. The data may then be or simultaneously be synchronized to the tracked position of the OCT probe such that the data may be spatially-resolved. This may be desirable as the data acquired by an OCT probe may generally or in some cases be of a relatively small region of the target and/or difficult to resolve spatially in relation to the target as a whole. The direction of scanning of the OCT probe may also be resolved to, for example, correlate the data gathered by the OCT probe.

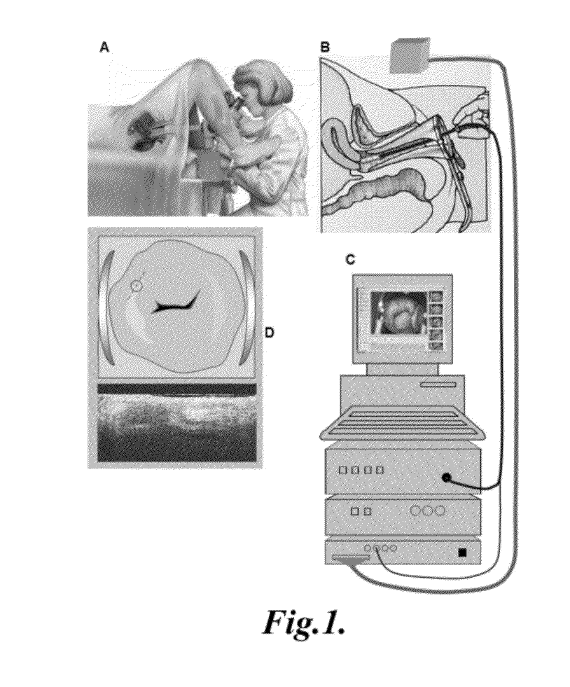

In one embodiment, as illustrated in FIG. 1, a spatially-resolved OCT system may be utilized, for example, for imaging the cervix. As illustrated, standard digital colposcopy may be performed to image the cervix. In (B), the colposcope may be removed, and an OCT probe may be introduced through the speculum for OCT imaging of the cervix. In (C), the OCT probe may include and/or have coupled to it instrumentation which may allow spatial localization relative to, for example, cervix, and a software application (D) may indicate the location and orientation of OCT images gathered by the OCT probe relative to cervical image(s) gathered by the colposcope. The system may also be utilized for any other appropriate application of OCT, or any appropriate application for a different sensing or imaging modality if employed. Other OCT probes may also be employed, which may include, but are not limited to gastrointestinal probes, intravascular probes, cardiac probes and/or any other appropriate probe.

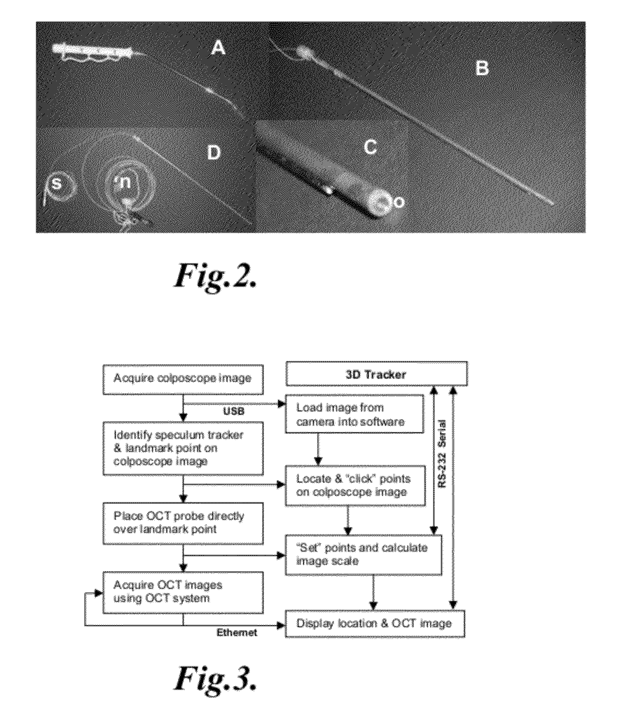

In some embodiments, an OCT probe may be, for example, a 2D scanning fiberoptic OCT imaging probe, such as from NIRIS (NIRIS imaging system, Imalux, Cleveland, Ohio), a ThorLabs swept-source OCT imaging system (OCS1300SS, ThorLabs, Edison, N.J.), and/or any other appropriate OCT or other probe/system. In one embodiment, as illustrated in FIG. 2, a tracking probe t, such as the TrackSTAR probe, may be coupled to an OCT probe o, such as the NIRIS probe.

In one exemplary aspect, the spatially-resolved data may be utilized to generate a three-dimensional (3D) dataset. This may be desirable as, for example, OCT probes typically gather two-dimensional (2D) data and it may be useful or desirable to resolve such data in 3D. In one exemplary embodiment, the tracking system and data acquisition system may collect and/or synchronize positioning data and OCT-gathered data temporally such that the data may be correlated to generate a spatially-resolved dataset, such as 3D dataset. It may also be desirable in some embodiments to utilize 2D probes as they may generally be smaller and/or less expensive than 3D probes, and the available field which may be acquired may be generally larger. 3D data from a 3D OCT probe may also be spatially resolved to generate a 3D dataset, such as from a manually-operated 3D OCT probe where resolution of the manual acquired data may be required to generate a usable dataset. The 3D dataset may further be utilized, for example, to generate a 3D graphical representation of the scanned target.

In another exemplary aspect, the tracking system may be utilized to guide acquisition of data from a 2D OCT probe to generate a 3D dataset. In one embodiment, the tracking system may, for example, indicate the region of a target for which data has been acquired. For example, the tracking system may indicate to a user what volume of space of the target for which data has been acquired which may enable the user to “fill in” the space during data acquisition to help ensure that data has been acquired for a desired volume. For example, though real-time reconstruction of OCT images may be difficult or in some cases not presently possible due to, for example, the computational complexity involved, it may be desirable to provide feedback to the user indicative of “how good” a manual scan is as it is performed. In some embodiments, a visualization technique in which a translucent “datacube” is “filled up”, as tracking data indicates that individual voxels have been acquired by an OCT probe that may be spatially tracked. The user may then “paint” the volume with the OCT probe such that complete and/or adequate coverage may be achieved for the construction of a 3D dataset.

### Example of a 3D-Tracked OCT Probe

FIG. 2 shows a real-time 3D-tracked endoscopic OCT probe. A TrackSTAR DC magnetic field six degree-of-freedom active position sensing system (trackSTAR, Ascension Technology, Burlington, Vt.) was coupled to a 2D scanning fiberoptic OCT imaging probe (NIRIS imaging system, Imalux, Cleveland, Ohio). A probe holder was constructed based on a non-ferromagnetic brass tube with bonded polyimide sleeve and Touhy-Borst locking adapter. The sensor was offset from the probe face by 22 mm to aid in eliminating interference. As shown in FIG. 2: (A) Probe sheath provided with Imalux device. (B) tracking probe created for sheep imaging studies. (C) close-up of distal end showing OCT probe face (o) and tracker (t). (D) entire probe with lead sensor (s) and fiber/wires for Niris probe (n).

### Example of Determining Positioning Accuracy

In bench top experiments, the accuracy of both the TrackStar sensor alone and in conjunction with the NIRIS OCT imaging probe were verified. Table 1 summarizes these findings:

| | | | | | | | Angle | | Angle | | Angle | |
|---|---|---|---|---|---|---|---|---|---|---|---|---|
| | Position | | Position | | Position | | Azi'th | | Elev'n | | Roll | |
| | X (inch) | | Y (inch) | | Z (inch) | | (degree) | | (degree) | | (degree | |
| | μ | σ | μ | σ | μ | σ | μ | σ | μ | σ | μ | σ |
| Bare | <.002 | .0023 | <.002 | .0018 | <.002 | .0013 | <.02 | .0071 | <.02 | .081 | .012 | <.02 |
| Sensor | | | | | | | | | | | | |
| Probe - | .721 | .0017 | −.326 | .0019 | 0.457 | .0013 | 6.32 | .0095 | 10.5 | .092 | .013 | −4.40 |
| Tip | | | | | | | | | | | | |
| Probe - | <.002 | .0021 | <.002 | .0022 | <.002 | .0014 | <.02 | .013 | <.02 | .077 | .016 | <.02 |
| Back | | | | | | | | | | | | |

Mean and Standard Deviation Measured for repeated transit of sensor between two fixed locations/orientations. Experiments were repeated “bare” tracking sensor (attached to brass tube) alone, sensor located adjacent to tip of NIRIS OCT probe, and Sensor located 22 mm rearward from NIRIS probe tip. Note that resolution of reported maximum result is 0.001″ and 0.01°. The NIRIS probe head contains a small but strong magnet (for scanning). Proximity to magnet caused significant distortion in gross measurement but was obviated when sensor was moved rearward a distance of approximately 2 cm.

Similar verification (not shown) was made for placement of a tracking sensor onto the blade of a stainless steel speculum for position tracking.

### Example of Software Design for Cervical Tissue Image Registration Techniques

The flowchart in FIG. 3 illustrates the communication and data/work flow for the registration software. A colposcope equipped with a digital SLR is used to acquire and image of the cervix in the usual way. The image is transferred by USB, and a tracker embedded in the speculum identified on the image. Additionally, any other landmark or point of interest can be specified as a second calibration point (for image scaling). Once identified on the image, the operator simply places the OCT device against the same landmark and presses “Set” button on the tracking computer. Subsequently, the 6-dof position of the OCT imaging probe is tracked in real-time. Because the orientation of the tracking sensor relative to the OCT probe face is known, rotation matrices are used to calculate the center and orientation of the probe face and project it onto the colposcopic image. The 6-dof sensor embedded in the speculum allows correction for subject motion. By “sharing” the data directory of the OCT acquisition computer over the network, the registration software can pull over new OCT images as soon as they are available and record their corresponding location.

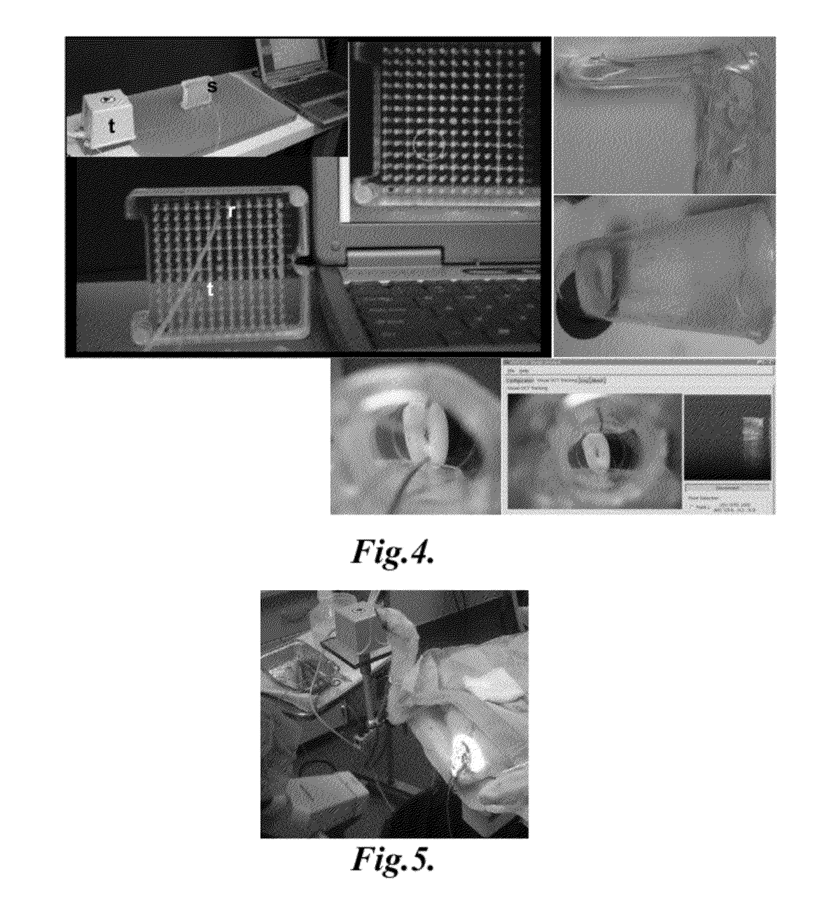

An experimental setup using a precision machined grid as was used to test the software by using various grid holes for calibration and measurement test points as shown in FIG. 4. Repeated trials indicated that the average absolute measurement error was less than 0.5 mm and the maximum absolute error was 1.5 mm. Apparent image location measurements were also small except near the edges of the image where perspective distortion in the photograph may play a larger role. To gain feedback from clinicians regarding a “look and feel” for the software, a cervix phantom was also utilized, as shown in FIG. 4. In FIG. 4: Experimental setup. (TOP) Transmitter (t) and single sensor (s) in grid with holes precisely on 5 mm centers. (ABOVE) Image of grid with reference (r) sensor and test (t) sensor and corresponding real-time registration on computer. (TOP RIGHT) Speculum with sensor holder and cervical phantom. (ADJACENT) Image of probe on “cervix” and corresponding software screen capture.

### Example of Animal Use

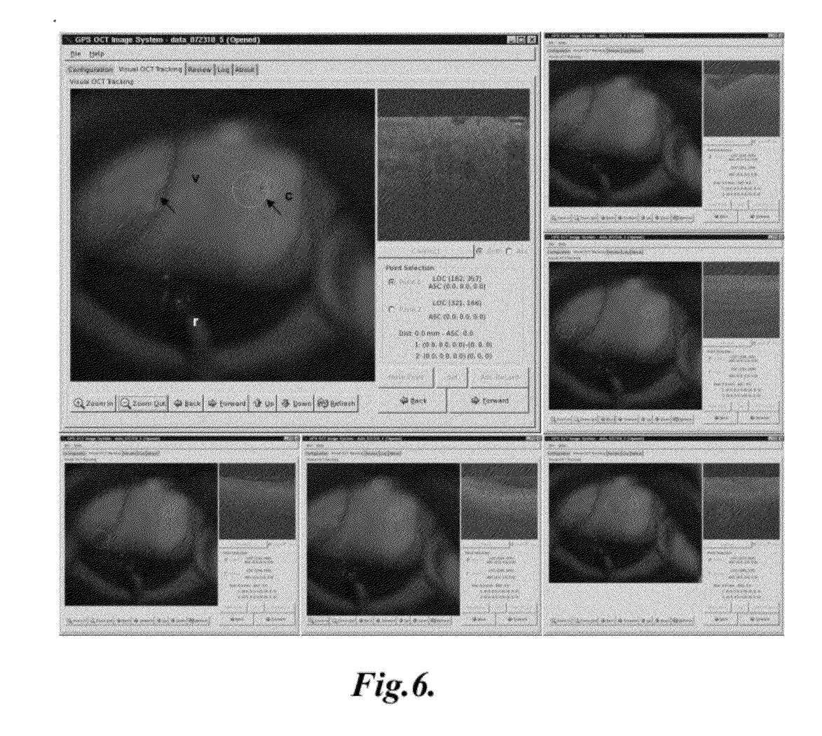

The OCT registration system was utilized with on-going OCT examinations of the endovaginal canal and cervix in sheep. In four virginal yearling Rambouillet sheep, an OCT probe holder with integrated tracker was used and affixed with a second reference tracker to the lower blade of the speculum. Animals were anesthetized and maintained with inhaled isoflurane for imaging. At the end of the planned survey of the animal, an additional colposcope image was obtained which included visualization of the reference sensor on the lower speculum blade. After the image was transferred, an identifiable landmark (for example the cervical os) was selected for calibration of the tracking sensor and image. Subsequently, several OCT images were acquired while the position and orientation of the OCT probe were tracked and displayed in real time, fused in registration to the colposcope image. One additional animal (FIG. 5) was obtained which had not been received the treatment being investigated, and electrocautery was used to create two small lesions on the cervix and vaginal wall as features for OCT imaging. Screen captures from this animal are shown in FIG. 6. In FIG. 6: Screen Captures from OCT Registration Software Package. Electrocautery lesions (black arrows) are seen in the colposcope image of cervix (c) and vaginal wall (v). Identification of reference tracker (r) at point 1 and lesion at point 2 allows tracking of probe position and orientation (green circle, crosshair) during imaging. As OCT images are acquired, they are displayed (Right of colposcope image) and ‘tagged’ with image coordinates for later review.

Example of Wide-Field Volume Set Reconstruction from Manually-Guided OCT Imaging

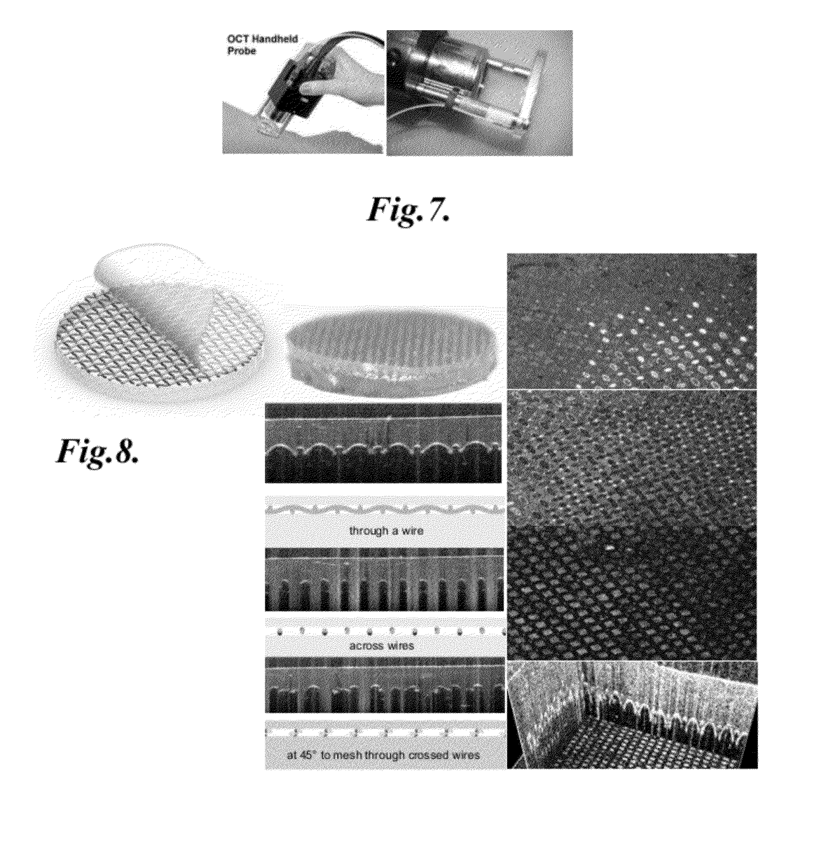

Real-time 3D tracking of a 2D imaging probe offers an exciting possibility of enabling 3D OCT image acquisitions with smaller 2D probes. Successful acquisition and reconstruction of 3D OCT images from spatially tracked 2D acquisitions was demonstrated. To do so, a commercially available swept-source OCT system operating at 1300 nm and capable of 2D and 3D acquisitions with 2D B-scan rates of up to 25 fps (OCS1300SS, ThorLabs, Edison, N.J.) was utilized. The system includes a hand-held imaging probe comprising X- and Y-scan galvos and a wide-field objective (FIG. 7). For tracking, we outfitted it with a 1.2 mm diameter 6-dof 3D guidance probe (FIG. 7). We also constructed a phantom made from a fine wire mesh embedded in a silicone wafer (FIG. 8). A number of OCT images through various aspects of this phantom are shown for comparison to subsequent “tracking-reconstructed” images. In FIG. 8: 3D OCT Imaging Phantom. (LEFT) CAD illustration and photograph of 13-mm diameter phantom. (FAR RIGHT) horizontal OCT “slices” through phantom at increasing depths (b/w images) and 3D rendering from OCT viewing software (orange). (CENTER) Several 2D OCT images through phantom and corresponding cut sections through the CAD representation.

### Example of Methodology for Reconstruction of 3D OCT Data

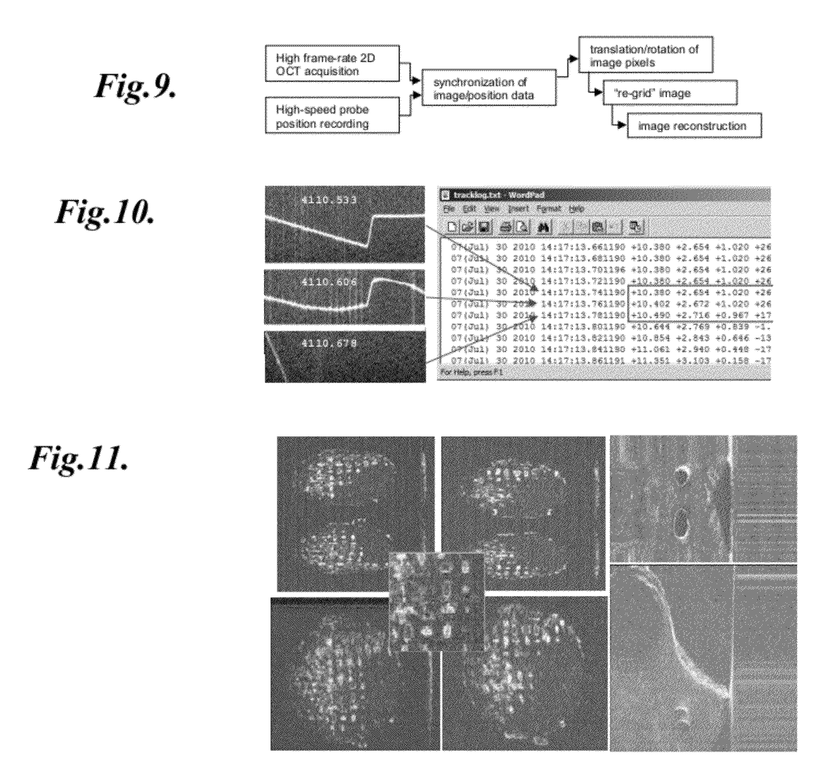

A general method for reconstruction of 3D OCT is illustrated in FIG. 9. From this flow-chart, several critical factors, for example, can be gleaned. First, the OCT acquisition rate and the position acquisition rate must be sufficient to capture/track movements on the order of the OCT feature resolution. Second, high-resolution synchronization between the OCT and position data must be available.

### Example of Determination of Time-Stamp Synchronization

The 2D (B-mode) OCT acquisition rate was at up to 25 Hz (25 fps). However, all acquisitions were made at half this rate (13 fps). Thus, a single OCT acquisition required approximately 77 ms. The trackSTAR tracker is capable of supplying fully updated measurement data at a rate of 80 Hz (80 measurements per second) or about 12 ms update time. However, because the trackSTAR was used with an RS-232 serial communication link, the communication speed limited the update rate to one measurement every 20 ms (50 Hz).

Because the OCT and trackSTAR data were acquired on separate computer systems, the clock signals were synchronized (and hence timestamps) between the OCT system (Windows XP) and trackSTAR software (Linux). An empirical method was employed in which the OCT probe was moved rapidly from a stationary position and synchronized changes observed in OCT frames and tracker output data were performed (FIG. 10). With multiple experimental replicates, synchronization of approximately 10-20 ms, or at about the resolution of the tracking measurements, was obtained. In FIG. 10: the OCT probe was rapidly moved away from a stable position, and image frame time-stamps sync'd with changes in position data file. Top-to-Bottom are ‘baseline’, ‘transition’, and ‘moved’ OCT images with time values in seconds. At right is portion of tracker log file.

### Example of Image Acquisition and Reconstruction

Datasets were acquired by manually scanning the OCT probe over the phantoms multiple times. After the time synchronization factor was found, a combination of C code and shell scripts were used to automatically extract OCT frames and time-stamps and re-register OCT image data, and extract a desired cut plane into an image file. Since the tracker data had an effective resolution of about 0.004″ (0.1 mm), the data were re-sampled at higher spatial frequency before reconstruction. For each entry in the position log, the OCT frame with the nearest timestamp was placed into a corresponding “bin” in the image cube. For a simplified case, in which only y-direction movement was employed, reconstruction of a 2000-frame image required approximately 10 seconds. Several examples from a back-and-forth scan are shown in FIG. 11. In FIG. 11: Reconstructed OCT images are shown. OCT images acquired from rapid 2D scans were acquired as the OCT probe was moved over a 5 mm silicone phantom containing a wire mesh. Real-time position data were used to “relocate” each to its proper location in the data-cube. Then en-face slices through the datacube were turned into images. (A) and (B) a horizontal slice through a collection of B-scans before re-registration. (C) and (D) Identical slice depth after re-registration. (INSET) Note that the outlines of individual wires in the mesh can be discerned (compare to FIG. 8). (E) and (F) Pre- and pos-registration images for a similar phantom with irregular edge and 6 mm and 1 mm punctures. Note that the operator “bumped” (and moved) the phantom at some point during acquisition leading to apparent ghosting.

In other embodiments, small-field imaging modalities such as, for example, laser scanning, confocal, and/or multi-photon microscopy may be utilized similarly to the OCT modality described above.

It will be appreciated by those of ordinary skill in the art that the present invention can be embodied in other specific forms without departing from the spirit or essential character hereof. The present description is therefore considered in all respects to be illustrative and not restrictive. The scope of the present invention is indicated by the appended claims, and all changes that come within the meaning and range of equivalents thereof are intended to be embraced therein.

1. A system for resolving optical coherence tomography (OCT) measurements comprising:
an OCT probe;
a spatially-resolvable probe in fixed relation to said OCT probe; and
a tracking system in communication with said spatially-resolvable probe;
wherein said tracking system tracks the location of said spatially-resolvable probe and correlates data gathered by said OCT probe with said location of said spatially-resolvable probe.

2. The system of claim 1, wherein said OCT probe and said spatially-resolvable probe are integrated.

3. The system of claim 1, wherein said OCT probe comprises a 2D OCT probe.

4. The system of claim 1, wherein said OCT probe comprises a 3D OCT probe.

5. The system of claim 1, wherein said spatially-resolvable probe comprises a magnetic field sensing probe.

6. The system of claim 5, wherein said tracking system comprises a magnetic field positioning system.

7. The system of claim 1, wherein said tracking system comprises a temporal tracking system for correlating time, a position of said OCT probe and data acquired by said OCT probe.

8. The system of claim 1, wherein said OCT probe is selected from the group consisting of cervical probes, intravascular probes, cardiac probes and gastrointestinal probes.

9. The system of claim 1, wherein said spatially-resolvable probe is resolvable in six degrees of freedom.

10. A method for generating 3D OCT data comprising:
scanning a 3D target space with a 2D OCT probe to generate 2D data, said 2D OCT probe being in fixed relation to a spatially-resolvable probe;
tracking the location of said spatially-resolvable probe during said scanning; and
constructing a 3D dataset by assigning said 2D data to a 3D space by utilizing said location of said spatially-resolvable probe.

11. The method of claim 10, wherein said scanning is performed manually.

12. The method of claim 10, further comprising temporally correlating the location of said spatially-resolvable probe to said 2D data.

13. The method of claim 11, wherein said scanning is performed manually by a user filling in a volume representation on a graphical interface.

14. The method of claim 10, further comprising generating a 3D graphical representation of said target space using said 3D dataset.

15. The method of claim 10, further comprising resolving the direction of scanning of said 2D OCT probe.

16. A method for generating 3D OCT data comprising:
scanning a 3D target space with a 3D OCT probe to generate 3D data, said 3D OCT probe being in fixed relation to a spatially-resolvable probe;
tracking the location of said spatially-resolvable probe during said scanning; and
constructing a 3D dataset by assigning said 3D data to a 3D space by utilizing said location of said spatially-resolvable probe.

17. The method of claim 16, wherein said scanning is performed manually.

18. The method of claim 16, further comprising temporally correlating the location of said spatially-resolvable probe to said 3D data.

19. The method of claim 17, wherein said scanning is performed manually by a user filling in a volume representation on a graphical interface.

20. The method of claim 16, further comprising generating a 3D graphical representation of said target space using said 3D dataset.
